(19) United States
(12) Patent Application Publication
(10) Pub. No.: US 2023/0371784 A1
(43) Pub. Date: Nov. 23, 2023

(54) LAPAROSCOPE

(71) Applicant: MMILR INC., Daejeon (KR)

(72) Inventor: Jin-Hee MOON, Sejong (KR)

(21) Appl. No.: 18/031,368

(22) PCT Filed: Nov. 19, 2020
(86) PCT No.: PCT/KR2020/016349

§ 371 Date: Apr. 12, 2023

(30) Foreign Application Priority Data
Oct. 12, 2020 (KR) 10-2020-0130981

(51) Int. Cl. A61B 1/00; A61B 1/313; A61B 1/04

(52) U.S. Cl. CPC A61B 1/00016; A61B 1/3132; A61B 1/00165; A61B 1/04; A61B 1/00096; A61B 1/00158

(57) ABSTRACT

A magnetically fixed laparoscope of the present invention, which is inserted into an abdominal cavity for imaging or surgery of an affected area and whose position is controlled by means of an external magnetic fixing unit, comprises: one or more flat-type antenna units connected to a rotation axis within the laparoscope; an internal hollow unit which can accommodate the flat-type antenna units; and an external hollow unit which can be inserted into the abdominal cavity where the affected area is located. As such, since it is possible to easily fold or unfold an antenna built into the laparoscope in the abdominal cavity, the present invention has the effect of enabling wireless control of the laparoscope and transmission and reception of data.

### TECHNICAL FIELD

The present disclosure relates to a laparoscope. In detail, the present disclosure relates to a magnetically-fixed type laparoscope of which an antenna for wireless communication can be unfurled or furled in a roll type.

### BACKGROUND ART

Laparoscopic surgery is a method of operating for the internal organs after boring a hole of 0.5 to 1 cm through the navel of a human body and then inserting a laparoscope for taking images in the abdominal cavity to minimize surgery scars. Doctors perform various surgeries using specifically designed laparoscopic instruments while seeing images of the inside of the abdominal cavity. Recently, laparoscopic surgery has been developed to the point where it can replace laparotomy, and has the advantage of being able to reduce risks and stress accompanying laparotomy and post-of recovery time.

When laparoscopic surgery is performed using a laparoscope, an optical module is mounted therein to obtain an image inside the abdominal cavity and the image obtained through the optical module is transmitted to a monitor in a wired or wireless type and output, whereby doctors can perform surgery on the basis of the image output on the monitor.

However, when a laparoscope is controlled from the outside in a wired type, there is a problem that, generally, it is required to bore several holes such as a laparoscope hole, a control hole, and a camera hole through the abdomen of a patient.

When several holes are bored through the abdomen of a patient, there is a problem that the risks accompanying surgery and the post-op recovery time may relatively increase.

### DISCLOSURE

### Technical Problem

An objective of the present disclosure is to solve all of the problems described above.

An objective of the present disclosure is to provide a laparoscope that is controlled in a wireless type to be able to minimize post-surgery scars on the abdomen of a patient in laparoscopic surgery.

Another objective of the present disclosure is to provide a laparoscope of which a wirelessly controlled antenna can be easily unfurled or stowed therein.

### Technical Solution

The characteristic configuration of the present disclosure for achieving the objectives of the present disclosure and achieving the characteristic effects of the present disclosure to be described below is as follows.

According to an embodiment of the present disclosure, a magnetically-fixed type laparoscope that is inserted into an abdominal cavity to take an image of a diseased part or perform surgery on a diseased part and of which a position is controlled by an external magnetic fixing unit, includes: a micro camera; a lens module disposed at an end of the laparoscope; an optical fiber configured to transmit light traveling from the lens module to the micro camera; an inner hollow member configured to accommodate the micro camera, the lens module, and the optical fiber in an internal space; one or more flat type antenna units connected to a rotary shaft in the laparoscope; and an outer hollow member being able to accommodate the flat type antenna unit therein, including the inner hollow member, and configured to be inserted into an abdominal cavity with the diseased part.

In an embodiment, the flat type antenna unit is stowed in a roll type on the rotary shaft in the outer hollow member and is unfurled flat outside the outer hollow member along an in-abdominal cavity abdominal wall by rotation of the rotary shaft.

In an embodiment, the flat type antenna unit is provided in a pair and the flat type antenna units are unfurled flat in opposite directions outside the outer hollow member with rotation of the rotary shaft.

In an embodiment, the outer hollow member has a first slit and a second slit, and the flat type antenna unit includes a first flat type antenna unit that is unfurled flat in a first direction along the in-abdominal cavity abdominal wall through the first slit and a second flat type antenna unit that is unfurled flat in a second direction, which is an opposite direction of the first direction, along the in-abdominal cavity abdominal wall through the second slit.

In an embodiment, the first flat type antenna unit has a stopper at an end thereof, so when the first flat type antenna unit is stowed in a roll type on the rotary shaft in the outer hollow member by rotation of the rotary shaft, the end is stopped and fixed on the first slit; and the second flat type antenna unit has a stopper at an end thereof, so when the second flat type antenna unit is stowed in a roll type on the rotary shaft in the outer hollow member by rotation of the rotary shaft, the end is stopped and fixed on the second slit.

In an embodiment, the first slit and the second slit are formed such that longitudinal directions thereof are parallel with each other outside the outer hollow member, and discharge directions of the slits to the outside from the inside of the outer hollow member are different from each other; and the first flat type antenna unit and the second flat type antenna unit are connected to one rotary shaft and are rotated in the same direction, and are unfurled in different directions through the first slit and the second slit.

In an embodiment, the first slit and the second slit are formed such that longitudinal directions thereof are parallel with each other outside the outer hollow member, and discharge directions of the slits to the outside from the inside of the outer hollow member are different from each other; and the first flat type antenna unit is connected to the rotary shaft and the second flat type antenna unit is connected to a rotary gear connected to the rotary shaft to rotate in an opposite direction of a rotation direction of the rotary shaft, so the first and second flat type antenna units can be unfurled in opposite directions through the first slit and the second slit, respectively, while rotating in opposite directions.

In an embodiment, when the flat type antenna is unfurled flat along the in-abdominal cavity abdominal wall, the unfurling flat type antenna is supported by an elastic member having a cross-section that has a recession when seen in a longitudinal direction of the outer hollow member.

In an embodiment, the rotary shaft is mechanically connected to an assistant inserter that pushes the laparoscope into the abdomen, and is rotated a predetermined angle in correspondence to control of the assistant inserter.

In an embodiment, the rotary shaft is a motor shaft that is electrically connected to an assistant inserter that pushes the laparoscope into the abdomen, and the motor is rotated a predetermined angle in correspondence to control of the assistant inserter.

In an embodiment, the outer hollow member has an insulating characteristic. Meanwhile, the flat type antenna includes a ground plane, a flexible substrate, and a rollable antenna element.

According to another aspect of the present disclosure, a magnetically-fixed type laparoscope that is inserted into an abdominal cavity to take an image of a diseased part or perform surgery on a diseased part and of which a position is controlled by an external magnetic fixing unit, includes: a micro camera; a lens module disposed at an end of the laparoscope; an optical fiber configured to transmit light traveling from the lens module to the micro camera; an inner hollow member configured to accommodate the micro camera, the lens module, and the optical fiber in an internal space; an outer hollow member including the inner hollow member and configured to be inserted into an abdominal cavity with the diseased part; and one or more film-type flat type antenna units having a center shaft accommodated in a seat formed in a housing of the outer hollow member, being able to move along a passage of the seat in the housing, and being unfurled while supporting an inner wall of the abdomen when the center shaft is moved outside from the inside of the seat.

In this configuration, the flat type antenna unit is composed of a pair symmetric antennas and the antennas can be unfurled in different directions while supporting the inner wall of the abdomen when the center shaft is moved.

Further, movement guides that can guide the antennas of the flat type antenna unit to be unfurled in different directions, respectively, are formed at an outside exit of the seat.

According to another aspect of the present disclosure, a magnetically-fixed type laparoscope that is inserted into an abdominal cavity to take an image of a diseased part or perform surgery on a diseased part and of which a position is controlled by an external magnetic fixing unit, includes: a micro camera; a lens module disposed at an end of the laparoscope; an optical fiber configured to transmit light traveling from the lens module to the micro camera; an inner hollow member configured to accommodate the micro camera, the lens module, and the optical fiber in an internal space; an outer hollow member including the inner hollow member and configured to be inserted into an abdominal cavity with the diseased part; and one or more flat type antenna units accommodated in a seat formed in a housing of the outer hollow member, and having a width corresponding to a depth of the seat.

In this case, the flat type antenna unit may be composed of a pair of symmetric antennas.

### Advantageous Effects

According to a laparoscope of the present disclosure, since an antenna disposed in the laparoscope can be easily unfurled and furled in an abdominal cavity, there is an effect that it is possible to wirelessly control the laparoscope and transmit/receive data.

Further, according to the present disclosure, it is possible to easily mechanically unfurl or furl a laparoscope antenna using an assistant inserter.

Further, according to the present disclosure, since it is possible to unfurl a built-in antenna in two directions from a laparoscope, it is possible to increase the performance of transmitting/receiving data to/from the outside.

Further, according to the present disclosure, there is an effect that even though a built-in antenna is unfurled, the antenna can maintain its shape through a concave elastic member.

### DESCRIPTION OF DRAWINGS

FIG. 1 schematically shows a laparoscope according to the present disclosure.

FIG. 2 is a cross-sectional view schematically showing the laparoscope according to the present disclosure.

FIG. 3 schematically shows that antennas of the laparoscope according to the present disclosure are furled or unfurled.

FIG. 4 shows that laparoscope antennas according an embodiment of the present disclosure have been stowed.

FIG. 5 shows that laparoscope antennas according to another embodiment of the present disclosure have been stowed.

FIG. 6 schematically shows a driving structure for the laparoscope antennas of FIG. 5.

FIG. 7 shows an elastic support of laparoscope antennas according to the present disclosure.

FIG. 8 is a cross-sectional view schematically showing a laparoscope according to another characteristic of the present disclosure.

FIG. 9 schematically shows that a pair of flat type antennas has been unfurled in the laparoscope shown in FIG. 8.

FIG. 10 is a cross-sectional view schematically showing a laparoscope according to another characteristic of the present disclosure.

### BEST MODE

The following detailed description of the present disclosure provides specific embodiments for achieving the present disclosure and refers to the accompanying drawings. The embodiments are described in detail so that those skilled in the art can achieve the present disclosure. It should be understood that various embodiments of the present disclosure are different, but do not need to be exclusive to each other. For example, specific shapes, structures, and characteristics described herein in relation to an embodiment may be achieved through other embodiments without departing from the spirit and scope of the present disclosure. Further, it should be understood that the positions or arrangement of components in embodiments may be changed without departing from the spirit and scope of the present disclosure. Accordingly, the following detailed description is not limitative and the scope of the present disclosure is, if appropriately described, limited only by the claims and equivalents of the claims. Like reference numerals indicate the same or similar functions in various terms in the drawings.

Hereafter, exemplary embodiments of the present disclosure are described in detail with reference to the accompanying drawings so that those skilled in the art can easily achieve the present disclosure.

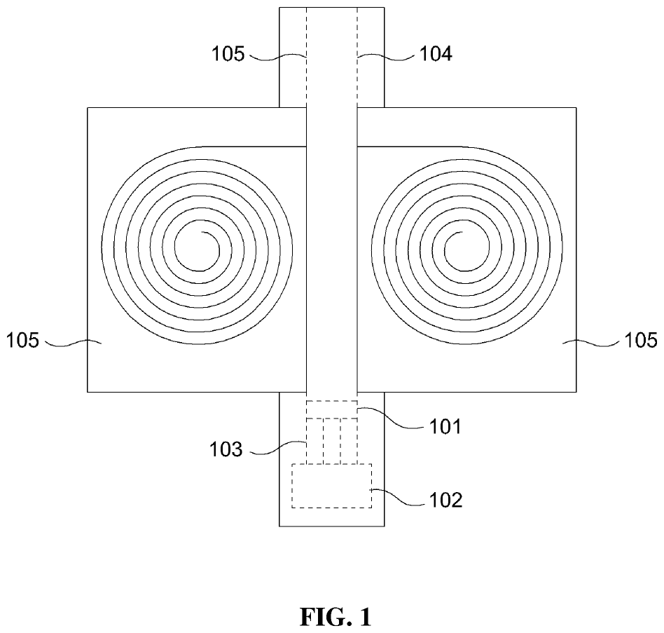

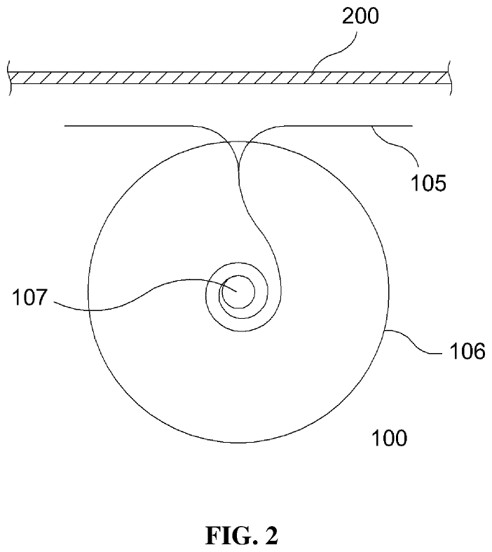

FIG. 1 schematically shows a laparoscope according to the present disclosure and FIG. 2 is a cross-sectional view schematically showing the laparoscope according to the present disclosure.

Referring to FIGS. 1 and 2, a laparoscope 100 according to the present disclosure may be a magnetically-fixed type laparoscope that is inserted into an abdomen to take images of a diseased part and perform surgery on the diseased part and of which the position is controlled by an external magnetic fixing unit or may be a general laparoscope that is directly connected to an external surgery control instrument through a cable or a pipe.

However, the laparoscope 100 according to an embodiment is configured to be wirelessly controlled or to wirelessly transmit/receive taking images, so it is preferable that the laparoscope 100 is a magnetically-fixed type laparoscope that is wirelessly connected and of which the position is controlled by magnetism rather than a wired-control type laparoscope.

The laparoscope 100 includes a micro camera 101, a lens module 102, an optical fiber 103, an inner hollow member 104, a flat type antenna unit 105, and an outer hollow member 106. The lens module 102 is disposed at an end of the laparoscope and the optical fiber 103 serves to transmit light traveling from the lens module 102 to the micro camera 101. The inner hollow member 104 serves to accommodate the micro camera 101, the lens module 102, the optical fiber cable, etc. in the internal space therein and the flat type antenna unit 105 functions as an antenna that is connected to an in-laparoscope rotary shaft 107 and can transmit or receive data to or from the outside. The outer hollow member 106 can accommodate the flat type antenna unit therein, includes the inner hollow member 104, and is configured to be inserted into an abdomen having a diseased part. Further, the outer hollow member 106 has an insulation characteristic.

Referring to FIG. 2, the flat type antenna unit 105 is stowed in a roll type on the rotary shaft 107 in the outer hollow member 106, and is unfurled flat outside the outer hollow member 106 along an in-abdominal cavity abdominal wall 200 by rotation of the rotary shaft 107. Preferably, the flat type antenna unit 105 is provided in a pair and can be unfurled flat in opposite directions outside the outer hollow member 106 with rotation of the rotary shaft 107. That is, as shown in FIG. 2, the flat type antenna unit 105 is provided in a pair at the left and right and can be unfurled in opposite directions from the laparoscope 100. The flat type antenna unit may be unfurled flat along the in-abdominal cavity abdominal wall 200.

The flat type antenna unit 105 may include a ground plane, a flexible substrate, and a rollable antenna element.

The rotary shaft 107 may be mechanically connected to an assistant inserter (not shown) that pushes the laparoscope 100 into the abdomen. That is, the laparoscope 100 and the assistant inserter are combined, the laparoscope 100 is inserted into the abdomen, the rotary shaft 107 is mechanically rotated a predetermined angle in correspondence to control of the assistant inserter, and the flat type antenna unit 105 stowed in a roll type is instantaneously unfurled with rotation of the rotary shaft 107. In another embodiment, the rotary shaft 107 may be a motor shaft that is electrically connected to the assistant inserter that pushes the laparoscope 100 into the abdomen. In this case, the motor is rotated a predetermined angle in correspondence to control of the assistant inserter, whereby the flat type antenna unit 105 stowed in a roll type is instantaneously unfurled.

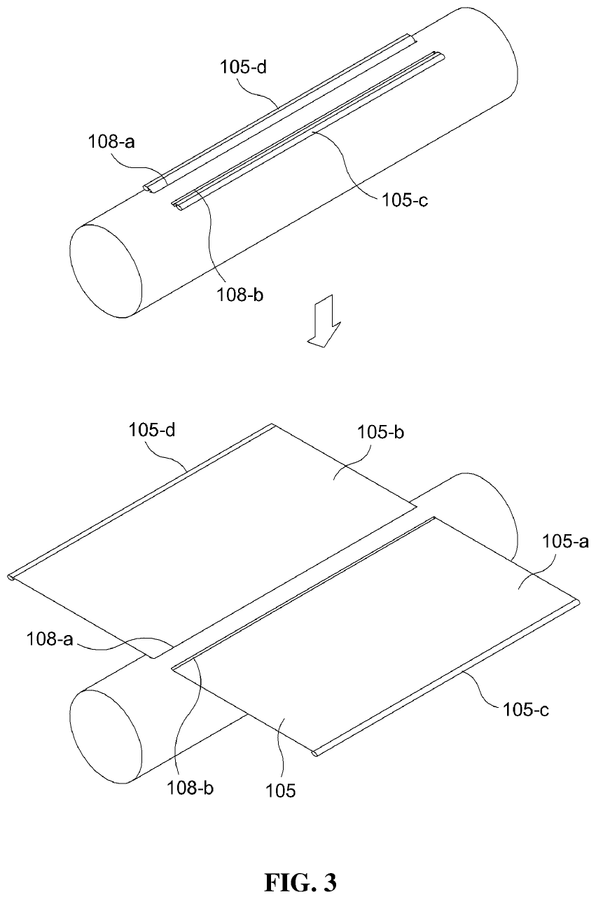

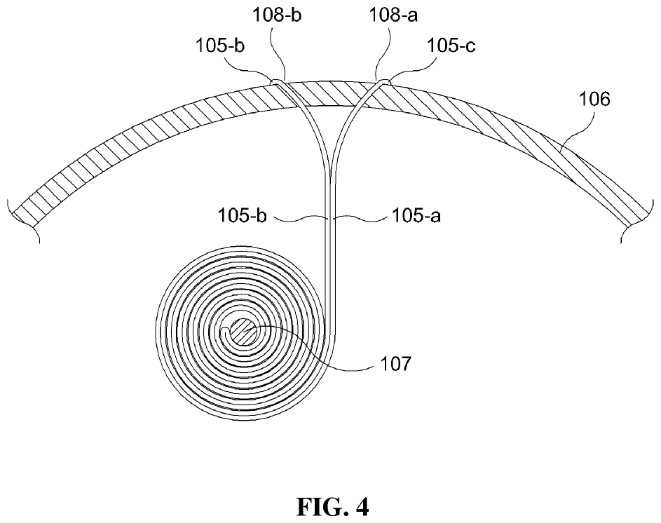

FIG. 3 schematically shows that antennas of the laparoscope according to the present disclosure are furled or unfurled and FIG. 4 shows that laparoscope antennas according an embodiment of the present disclosure have been stowed.

Referring to FIGS. 3 and 4, the outer hollow member 106 has a first slit 108_a and a second slit 108_b. The first slit 108_a and the second slit 108_b are disposed parallel with each other outside the laparoscope 100. The flat type antenna unit 105 includes a first flat type antenna unit 105_a that is unfurled flat in a first direction along the in-abdominal cavity abdominal wall 200 through the first slit 108_a and a second flat type antenna unit 105_b that is unfurled flat in a second direction along the in-abdominal cavity abdominal wall 200 through the second slit 108_b. It is preferable that the second direction is the opposite direction of the first direction.

Referring to FIG. 4, the first flat type antenna unit 105_a has a stopper 105c at the end, so when the first flat type antenna unit 105_a is stowed in a roll type on the rotary shaft 107 in the outer hollow member 106 by rotation of the rotary shaft 107, the end is stopped and fixed on the first slit 108a. Further, the second flat type antenna unit 105_b has a stopper 105d at the end, so when the second flat type antenna unit 105_b is stowed in a roll type on the rotary shaft 107 in the outer hollow member 106 by rotation of the rotary shaft 107, the end is stopped and fixed on the second slit 108_b.

Referring to FIG. 4 again, the first slit 108_a and the second slit 108_b are formed such that the longitudinal directions thereof are parallel with each other outside the outer hollow member, and the discharge directions of the slits to the outside from the inside of the outer hollow member 106 are different from each other. That is, it is preferable that the ends of the first slit 108_a and the second slit 108_b are formed in opposite directions so that the first flat type antenna unit 105_a and the second flat type antenna unit 105_b can be discharged in opposite directions. To this end, the discharge directions of the first slit 108_a and the second slit 108_b may be defined to draw arcs in opposite directions. The first flat type antenna unit 105_a and the second flat type antenna unit 105_b are connected to one rotary shaft 107 and are rotated in the same direction, whereby they are unfurled in opposite directions while stretching in the discharge directions of the first slit 108_a and the second slit 108_b.

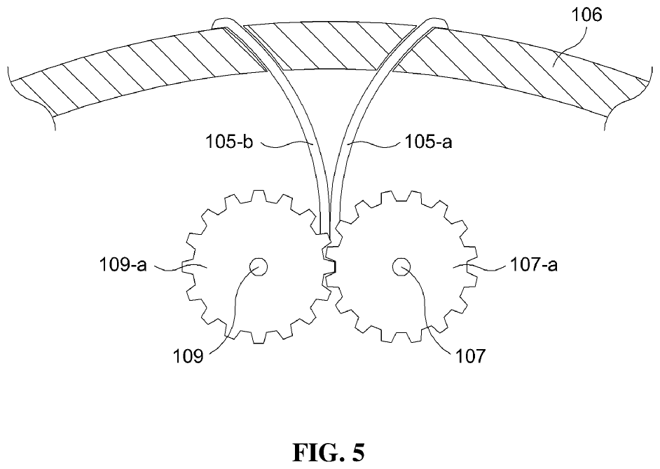

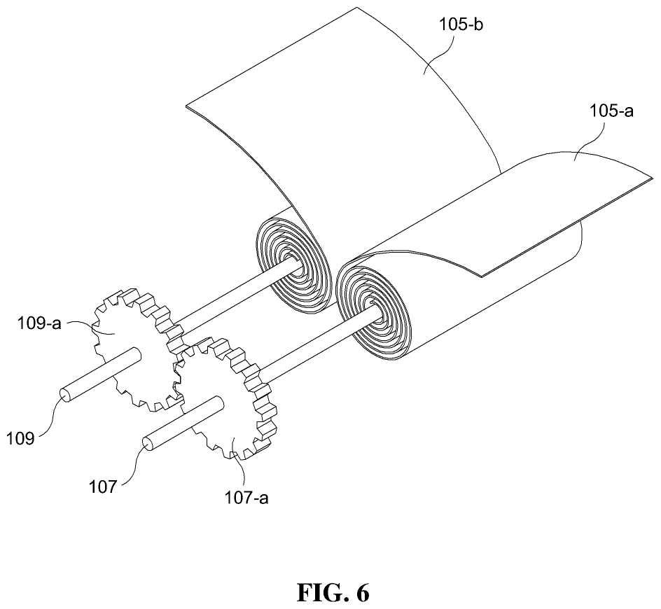

FIG. 5 shows that laparoscope antennas according to another embodiment of the present disclosure have been stowed. FIG. 6 schematically shows a driving structure of the laparoscope antennas of FIG. 5.

Referring to FIGS. 5 and 6, first slit 108_a and the second slit 108_b are formed such that the longitudinal directions thereof are parallel with each other outside the outer hollow member, and the discharge directions of the slits to the outside from the inside of the outer hollow member 106 are different from each other, which is the same as the example of FIG. 4. That is, in the embodiment of FIGS. 5 and 6 too, the ends of the first slit 108_a and the second slit 108_b are formed in opposite directions so that the first flat type antenna unit 105_a and the second flat type antenna unit 105_b can be discharged in opposite directions.

However, in the embodiment of FIGS. 5 and 6, the first flat type antenna unit 105_a is connected to the rotary shaft 107 and the second flat type antenna unit 105_b is connected to a rotary gear 107_a connected to the rotary shaft 107 to rotate in the opposite direction of the rotation direction of the rotary shaft 107, so the antenna units are rotated in opposite directions along a rotary shaft 109 of a rotary gear 109_b rotating in opposite directions. Accordingly, the first flat type antenna unit 105_a and the second flat type antenna unit 105_b can be rotated in opposite directions with rotation of the rotary shaft 107 and can be unfurled in opposite directions through the first slit 108_a and the second slit 108_b.

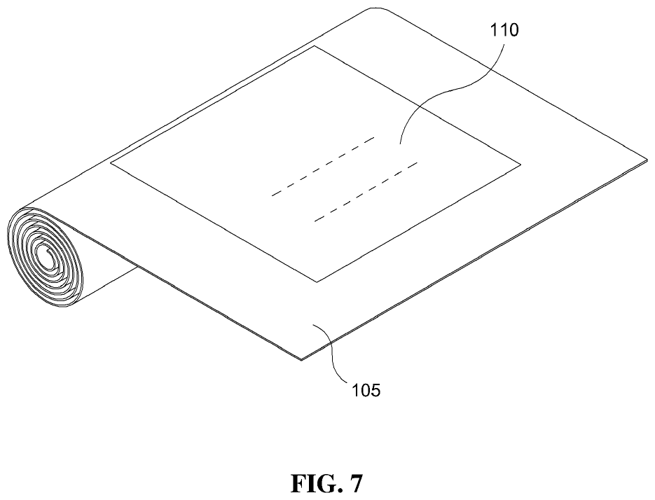

FIG. 7 shows an elastic support of laparoscope antennas according to the present disclosure.

Referring to FIG. 7, the flat type antenna 105 may include an elastic member 110, which has a concave cross-section, on the surface thereof. Accordingly, when the flat type antenna 105 is unfurled flat along an in-abdominal cavity abdominal wall, the unfurling flat type antenna can be supported by the elastic member 110 having a cross-section that has a recession when seen in the longitudinal direction of the outer hollow member. The elastic member 110 having a recession has a shape of which the center portion bends down and both ends bend up when seen in the longitudinal direction of a laparoscope, thereby supporting the flat type antenna 105 to prevent the flat type antenna 105 from sagging when being unfurled.

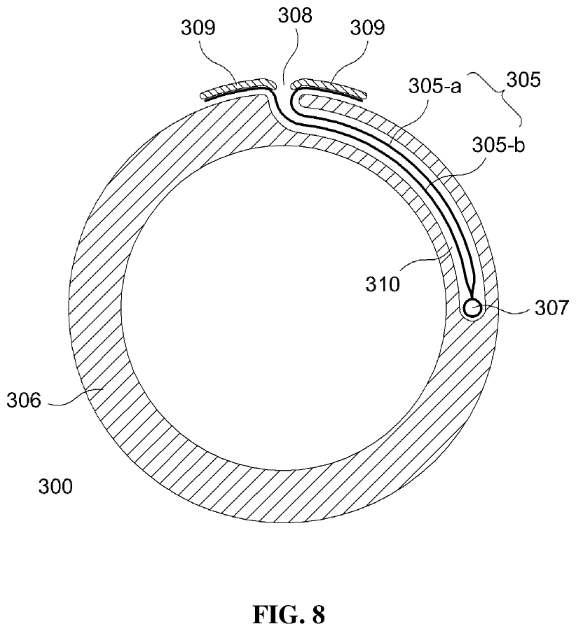

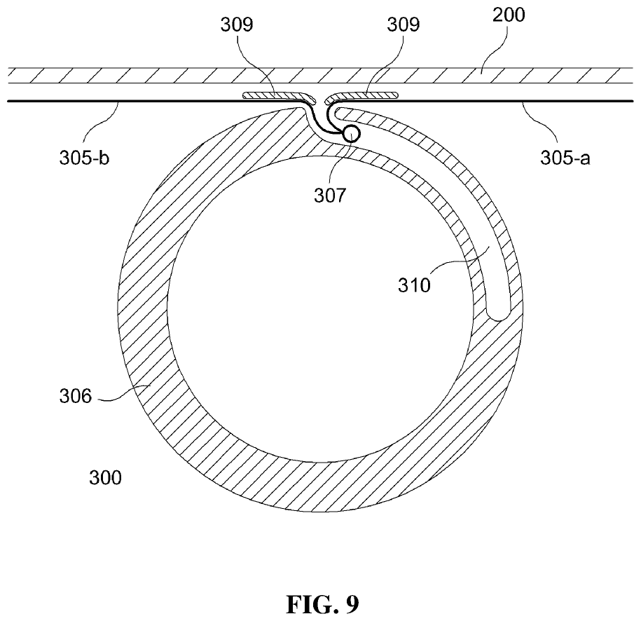

FIG. 8 is a cross-sectional view schematically showing a laparoscope according to another characteristic of the present disclosure. FIG. 9 schematically shows that a pair of flat type antennas has been unfurled in the laparoscope shown in FIG. 8.

Referring to FIGS. 8 and 9, a laparoscope 300 according to another characteristic of the present disclosure, unlike the laparoscope 100 shown in FIGS. 1 to 7, is characterized in that a flat type antenna 305 is stowed in a seat 310 formed in an outer hollow member housing of the laparoscope 300.

A center shaft 307 of the flat type antenna unit 305 is accommodated in the seat 310, so the flat type antenna unit 305 can be moved along the shape of the seat 310, that is, through a seat passage. That is, the center shaft 307 can be moved from the innermost side of the seat 310 to the vicinity of an outside exit 308. When the center shaft 307 is accommodated at the innermost side of the seat 310, the flat type antenna unit 305 is stowed in the seat 310 in the housing of the outer hollow member 306, and when the center shaft 307 is moved to the vicinity of the outside exit 308 of the seat 310, the film-type flat type antenna unit 305 is unfurled while supporting the in-abdominal cavity abdominal wall 200. The flat type antenna unit 305 is composed of a pair of antennas 305_a and 305_b, and these antennas are unfurled in opposite directions while supporting the in-abdominal cavity abdominal wall 200 when the center shaft 307 is moved. Meanwhile, movement guides 309 that can guide the pair of antennas 305_a and 305_b being unfurled in opposite directions are formed at the outside exit 308 of the seat 310.

The guides 309 may be separated in two directions at the exit 308 or may be independently formed on the outer wall of the outer hollow member 306. It is preferable that the ends of the guides 309 bend toward the outer hollow member 306 to prevent the flat type antenna unit 305 from winding upward or bending in the opposite direction.

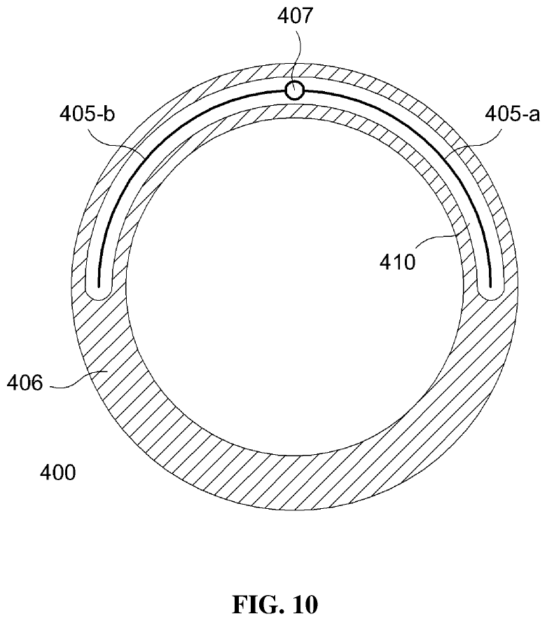

FIG. 10 is a cross-sectional view schematically showing a laparoscope according to another characteristic of the present disclosure.

A laparoscope 400 shown in FIG. 10 is configured such that flat type antenna units 405_a and 405_b are disposed and fixed in a seat 410 formed in a housing of an outer hollow member 406. The flat type antenna units 405_a and 405_b are preferably formed to have a width corresponding to the depth of the seat 410 and may be formed to surround the outer hollow member 406 in opposite directions by being unfurled in two directions from a center shaft 407.

As described above, according to a laparoscope of the present disclosure, since an antenna disposed in the laparoscope can be easily unfurled and furled in an abdominal cavity, there is an effect that it is possible to wirelessly control the laparoscope and transmit/receive data. Further, it is possible to easily mechanically unfurl or furl a laparoscope antenna using an assistant inserter. Further, according to the present disclosure, since it is possible to unfurl a built-in antenna in two directions from a laparoscope, it is possible to increase the performance of transmitting/receiving data to/from the outside, and there is an effect that even though a built-in antenna is unfurled, the antenna can maintain its shape through a concave elastic member.

Embodiments of the present disclosure described above may be implemented in the type of program commands the can be executed through various computer components, and may be recorded on a computer-readable recording medium. The computer-readable recording medium may include program commands, data files, and data structures individually or in combinations thereof. The program commands that are recorded on a computer-readable recording medium may be those specifically designed and configured for the present disclosure or may be those available and known to those engaged in computer software in the art. The computer-readable recording medium includes magnetic media such as hard disks, floppy disks, and magnetic media such as a magnetic tape, optical media such as CD-ROMs and DVDs, magneto-optical media such as floptical disks, and hardware devices specifically configured to store and execute program commands, such as ROM, RAM, and flash memory. The program commands include not only machine language codes compiled by a compiler, but also high-level language code that can be executed by a computer using an interpreter etc. The hardware device may be configured to operate as one or more software modules to perform the processing according to the present disclosure, and vice versa.

Hereinabove, although the present disclosure is described by specific matters such as concrete components, and the like, embodiments, and drawings, they are provided only for assisting in the entire understanding of the present disclosure. Therefore, the present disclosure is not limited to the embodiments. Various modifications and changes may be made by those skilled in the art to which the present disclosure pertains from this description.

Therefore, the sprit of the present disclosure should not be limited to the above-described embodiments, and the following claims as well as all modified equally or equivalently to the claims are intended to fall within the scope and spirit of the disclosure.

1. A magnetically-fixed type laparoscope that is inserted into an abdominal cavity to take an image of a diseased part or perform surgery on a diseased part and of which a position is controlled by an external magnetic fixing unit, the laparoscope comprising:
a micro camera;
a lens module disposed at an end of the laparoscope;
an optical fiber configured to transmit light traveling from the lens module to the micro camera;
an inner hollow member configured to accommodate the micro camera, the lens module, and the optical fiber in an internal space;
one or more flat type antenna units connected to a rotary shaft in the laparoscope; and
an outer hollow member being able to accommodate the flat type antenna unit therein, including the inner hollow member, and configured to be inserted into an abdominal cavity with the diseased part.

2. The laparoscope of claim 1, wherein the flat type antenna unit is stowed in a roll type on the rotary shaft in the outer hollow member and is unfurled flat outside the outer hollow member along an in-abdominal cavity abdominal wall by rotation of the rotary shaft.

3. The laparoscope of claim 2, wherein the flat type antenna unit is provided in a pair and the flat type antenna units are unfurled flat in opposite directions outside the outer hollow member with rotation of the rotary shaft.

4. The laparoscope of claim 3, wherein the outer hollow member has a first slit and a second slit, and
the flat type antenna unit includes a first flat type antenna unit that is unfurled flat in a first direction along the in-abdominal cavity abdominal wall through the first slit and a second flat type antenna unit that is unfurled flat in a second direction, which is an opposite direction of the first direction, along the in-abdominal cavity abdominal wall through the second slit.

5. The laparoscope of claim 4, wherein the first flat type antenna unit has a stopper at an end thereof, so when the first flat type antenna unit is stowed in a roll type on the rotary shaft in the outer hollow member by rotation of the rotary shaft, the end is stopped and fixed on the first slit; and the second flat type antenna unit has a stopper at an end thereof, so when the second flat type antenna unit is stowed in a roll type on the rotary shaft in the outer hollow member by rotation of the rotary shaft, the end is stopped and fixed on the second slit.

6. The laparoscope of claim 4, wherein the first slit and the second slit are formed such that longitudinal directions thereof are parallel with each other outside the outer hollow member, and discharge directions of the slits to the outside from the inside of the outer hollow member are different from each other; and
the first flat type antenna unit and the second flat type antenna unit are connected to one rotary shaft and are rotated in the same direction, and can be unfurled in different directions through the first slit and the second slit.

7. The laparoscope of claim 4, wherein the first slit and the second slit are formed such that longitudinal directions thereof are parallel with each other outside the outer hollow member, and discharge directions of the slits to the outside from the inside of the outer hollow member are different from each other; and
the first flat type antenna unit is connected to the rotary shaft and the second flat type antenna unit is connected to a rotary gear connected to the rotary shaft to rotate in an opposite direction of a rotation direction of the rotary shaft, so the first and second flat type antenna units can be unfurled in opposite directions through the first slit and the second slit, respectively, while rotating in opposite directions.

8. The laparoscope of claim 2, wherein when the flat type antenna is unfurled flat along the in-abdominal cavity abdominal wall, the unfurling flat type antenna is supported by an elastic member having a cross-section that has a recession when seen in a longitudinal direction of the outer hollow member.

9. The laparoscope of claim 1, wherein the rotary shaft is mechanically connected to an assistant inserter that pushes the laparoscope into the abdomen, and is rotated a predetermined angle in correspondence to control of the assistant inserter.

10. The laparoscope of claim 1, wherein the rotary shaft is a motor shaft that is electrically connected to an assistant inserter that pushes the laparoscope into the abdomen, and the motor is rotated a predetermined angle in correspondence to control of the assistant inserter.

11. The laparoscope of claim 1, wherein the outer hollow member has an insulating characteristic.

12. The laparoscope of claim 1, wherein the flat type antenna unit includes a ground plane, a flexible substrate, and a rollable antenna element.

13. A magnetically-fixed type laparoscope that is inserted into an abdominal cavity to take an image of a diseased part or perform surgery on a diseased part and of which a position is controlled by an external magnetic fixing unit, the laparoscope comprising:
a micro camera;
a lens module disposed at an end of the laparoscope;
an optical fiber configured to transmit light traveling from the lens module to the micro camera;
an inner hollow member configured to accommodate the micro camera, the lens module, and the optical fiber in an internal space;
an outer hollow member including the inner hollow member and configured to be inserted into an abdominal cavity with the diseased part; and
one or more film-type flat type antenna units having a center shaft accommodated in a seat formed in a housing of the outer hollow member, being able to move along a passage of the seat in the housing, and being unfurled while supporting an inner wall of the abdomen when the center shaft is moved outside from the inside of the seat.

14. The laparoscope of claim 13, wherein the flat type antenna unit is composed of a pair symmetric antennas and the antennas are unfurled in different directions while supporting the inner wall of the abdomen when the center shaft is moved.

15. The laparoscope of claim 14, wherein movement guides that can guide the antennas of the flat type antenna unit to be unfurled in different directions, respectively, are formed at an outside exit of the seat.

16. A magnetically-fixed type laparoscope that is inserted into an abdominal cavity to take an image of a diseased part or perform surgery on a diseased part and of which a position is controlled by an external magnetic fixing unit, the laparoscope comprising:
a micro camera;
a lens module disposed at an end of the laparoscope;
an optical fiber configured to transmit light traveling from the lens module to the micro camera;
an inner hollow member configured to accommodate the micro camera, the lens module, and the optical fiber in an internal space;
an outer hollow member including the inner hollow member and configured to be inserted into an abdominal cavity with the diseased part; and
one or more flat type antenna units accommodated in a seat formed in a housing of the outer hollow member, and having a width corresponding to a depth of the seat.

17. The laparoscope of claim 16, wherein the flat type antenna unit is composed of a pair of symmetric antennas.
